(19) United States
(12) Patent Application Publication
(10) Pub. No.: US 2010/0210848 A1
(43) Pub. Date: Aug. 19, 2010

(54) PROCESS FOR OPTICALLY ACTIVE SULFOXIDE COMPOUNDS

(75) Inventors: Ashok Kumar, Mumbai (IN); Dharmendra Singh, Thane (IN); Nellithanath Thankachen Byju, Mumbai (IN); Prasad Shankar Kadam, Mumbai (IN); Harishankar Prahladkumar Vishwakarma, Mumbai (IN); Vijay Ojha, Mumbai (IN); Umeshkumar Suresh Ninawe, District Bhandara (IN)

Correspondence Address: BLANK ROME LLP, WATERGATE, 600 NEW HAMPSHIRE AVENUE, N.W., WASHINGTON, DC, 20037, US

(73) Assignee: IPCA Laboratories Ltd., Mumbai (IN)

(21) Appl. No.: 12/681,386

(22) PCT Filed: Oct. 3, 2008
(86) PCT No.: PCT/IN08/00637

§ 371 Date: Apr. 2, 2010

(30) Foreign Application Priority Data
Oct. 3, 2007 (IN) 1967/MUM/2007
Oct. 3, 2007 (IN) 1968/MUM/2007
Oct. 3, 2007 (IN) 1969/MUM/2007

(51) Int. Cl. C07D 401/12

(52) U.S. Cl. 546/273.7

(57) ABSTRACT

The present invention discloses novel processes for preparing optically active sulphoxide compounds of formula I by asymmetric oxidation of prochiral sulphide compounds of Formula II. More particularly, the invention discloses processes for preparation of optically active proton pump Inhibitors (PPIs) or their optically active precursor (=intermediate) compounds (Formula I) that can be converted into pharmaceutically useful PPIs.

### FIELD OF THE INVENTION

The present invention relates to novel processes for preparing optically active sulphoxide compounds. More particularly, the invention relates to processes for optically active proton pump Inhibitors (PPIs) or their optically active precursor (=intermediate) compounds (Formula I) that can be converted into pharmaceutically useful PPIs.

### BACKGROUND OF THE INVENTION

Sulphoxide compounds, particularly, Pyridinylmethylsulphinyl benzimidazoles compounds of the following structure are known to have H+/K+-ATPase-inhibitory action and therefore have considerable importance in the therapy of diseases associated with an increased secretion of gastric acid or used as anti-ulcerative agent. Many sulphoxide compounds of closely related structure are known, for example, from EP0005129, EP166287, EP174726 and EP268956.

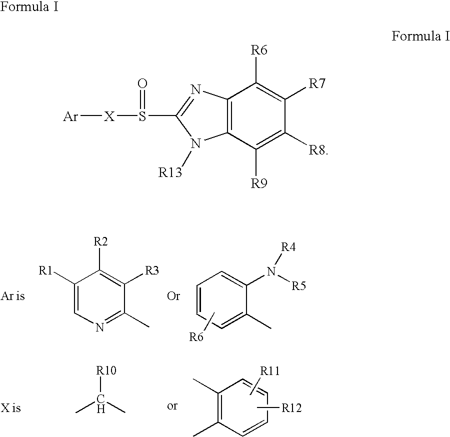

wherein R1, R2 and R3 are the same or different and selected from hydrogen, halogen, nitro, alkyl, alkylthio, alkoxy optionally substituted by fluorine, alkoxyalkoxy, dialkylamino, piperidino, morpholino, halogen, phenylalkyl and phenylalkoxy; R4 and R5 are the same or different and selected from hydrogen, alkyl and aralkyl; R6′ is hydrogen, halogen, trifluoromethyl, alkyl or alkoxy; R6-R9 are the same or different and selected from hydrogen, alkyl, alkoxy, halogen, halo-alkoxy, alkylcarbonyl, alkoxycarbonyl, oxazolyl, trifluoroalkyl, or adjacent groups R6-R9 form ring structures which may be further substituted; R10 is hydrogen or forms an alkylene chain together with R3 and R11 and R12 are the same or different and selected from hydrogen, halogen and alkyl; R13 is hydrogen or a protective group such as benzyl, trityl etc.

In the above definitions alkyl groups, alkoxy residues may be branched or straight C1-C9-chains or comprise cyclic alkyl groups, for example cycloalkylalkyl.

Examples of pharmaceutically active PPIs falls with in the compounds of Formula I, are 5-methoxy-2-[(4-methoxy-3,5-dimethyl-2-pyridinyl)methylsulphinyl]-1H-benzimidazole (also named as omeprazole), (S)-5-methoxy-2-[(4-methoxy-3,5-dimethyl-2-pyridinyl)methylsulphinyl]-1H-benzimidazole (common name: esomeprazole), 5-difluoromethoxy-2-[(3,4-dimethoxy-2-pyridinyl)methylsulphinyl]-1H-benzimidazole (Common name: pantoprazole), 2-[3-methyl-4-(2,2,2-trifluoroethoxy)-2-pyridinyl)methylsulphinyl]-1H-benzimidazole (Common name: lansoprazole), 2-{[4-(3-methoxypropoxy)-3-methylpyridin-2-yl]methylsulphinyl}-1H-benzimidazole (rabeprazole) and 5-methoxy-2-((4-methoxy-3,5-dimethyl-2-pyridylmethyl)sulphinyl)-1H-imidazo (4,5-b) pyridine (tenatoprazole).

The above-mentioned sulphoxide compounds are also referred to as proton pump inhibitors or abbreviated PPI owing to their mechanism of action. These compounds are chiral because of generation of chirality at sulphur atom when a prochiral sulphide is oxidized to sulphoxide, and therefore exists in two enantiomeric forms, namely the dextrorotatory isomer and Levorotatory isomer, which is also symbolized as R-isomer and the S-isomer. The process conventionally used for preparing the sulphoxide is the oxidation of the corresponding prochiral sulphides leading to a racemic mixture comprising about the same proportions of the two enantiomers, i.e. the (+)- and (−)-form or the (R)- and (S)-form of the sulphoxide compound.

Optical isomers of above sulphoxide compounds are known to have better efficacy or advantages in administration or helpful in reducing the dose regimen and therefore, of interest to a pharmaceutical chemist.

The preparation of optically active sulphoxide compounds are known from W091/12221, which describes a process for separating enantiomers using a cellulase enzyme. One of the active compounds illustrated in this process includes omeprazole.

A chemical process by classical racemate resolution was reported in W092/08716 that describes the separation of pyridin-2-ylmethylsulphinyl-1H-benzimidazole compounds into their optical isomers by formation of chiral diasteromeric derivatives. Compounds so prepared includes (+)- and (−)-5-difluoromethoxy-2-[(3,4-dimethoxy-2-pyridinyl)methylsulphinyl]-1H-benzimidazole[(+)- and (−)-pantoprazole] and (R) or (S)-omeprazole. A similar resolution process is disclosed in W094/27988 for the separation of racemic omeprazole into the enantiomers, using chiral auxiliaries and isolation of various pharmaceutical salts.

These methods suffer from various drawbacks, as they lead to loss of more than 50% of the unwanted isomer in the resolution process itself. The increased number of processing steps further escalates the cost and also the acid labile nature of target sulfoxides reduces the applicability for large scale application and therefore, such processes are not suitable for industrial production of pure optically active sulphoxide compounds.

Interestingly, U.S. Pat. No. 5,948,789 patent describes a process for the enantioselective synthesis of PPI using chiral titanium complexes, which is referred to as an improvement over the well known asymmetric oxidation processes of prochiral sulphides developed by Kagan et al. Kagan et al (please refer to J. Am. Chem. Soc. 106 (1984), 8188, or its improved version in Euro. J. Biochem. 166 (1987) 453) described a process for asymmetric oxidation of prochiral sulphides in presence of a chiral titanium complex (made of titanium derivative and a chiral ligand such as diethyl tartarate) in an organic solvent.

But according to the description of U.S. Pat. No. 5,948,789, even the Kagan's improved version of asymmetric oxidation of sulphides comprising a system of Ti(O-iPr)4/diethyl tartrate/water (1:2:1) in methylene chloride was reported to give very poor enantioselectivity/no selectivity for PPI's like Esomeprazole.

Therefore, in U.S. Pat. No. 5,948,789 patent, the enantioselectivity of oxidation of prochiral sulfides, especially for PPIs, was improved by conducting the oxidation using the same reactants/reagents in presence of a base in an organic solvent.

Similar processes are disclosed in WO/1999/025711, US20050187256 & WO2005054228 for sulphoxide compounds or its intermediate including esomeprazole. All these reactions were performed in an organic solvent. The enantioselective sulphoxidation for preparing esomeprazole on a large scale using a chiral titanium complex is also described in Tetrahedron, Asymmetry, (2000), 11, 3819-3825.

The enantioselective sulphoxidation of prochiral sulphides is extended to other chiral metal ligands like chiral Zirconium or vanadium complexes in J. Org. Chem., (1999), 64 (4), 1327 & WO/2004/052882.

A further process is disclosed in WO2006040685, by conducting the oxidation of sulphides in the presence of a chiral titanium complex & base in the absence of solvent. According to the description of '685 patent the oxidation reaction is conducted in anhydrous conditions. However the yields reported are very low, for example only about 30-32% with an enantioselectivity of about 92%, clearly indicating that carrying out the reaction in the absence of solvent does not improve the overall efficiency of the process for PPIs.

In the applicants' hands, except for the asymmetric oxidation in presence of a base and in an organic solvent, the chiral purity obtained in the above described processes is very poor or not applicable for industrial practice.

Apart from achieving the enantioselectivity, however, the most common problem with the above processes are the simultaneous formation of relative amounts of sulphone of formula III during reaction. This is due to the oxidation of the product sulfoxides (Formula I) during the reaction and no control method is available.

The formation of sulfones of formula (III) due to over-oxidation is almost impossible to avoid and this impurity is formed in very significant amounts, especially in the case of enantioselective oxidation processes, rather than conventional oxidation leading to a racemic product. There are some alternative solutions, such as performing the oxidation reaction at a very low temperature or lowering the amount of oxidizing agent were proposed to reduce the sulfone formation. But these alternatives are also reducing the efficacy of the oxidation reaction, and usually the amount of oxidizing agent or the reaction temperature are parameters affecting maximum conversion of starting material, maximum formation of sulfoxides of formula (I), overall enantioselectivity and overall product yield; and therefore one cannot compromise on these parameters.

It is being observed that the enantioselective oxidation processes described above, invariably leads to the formation of about 2 to 8% of the corresponding sulfone derivative (please refer to exemplary procedures of U.S. Pat. No. 5,948,789 or the comparative examples presented with this application), an over oxidized product. There is various purification processes described in the art, but the very similar physico-chemical characteristics of the product sulfoxide (Formula I) and sulfone impurity (Formula III) renders it difficult to purify and to obtain high quality sulfoxide (formula I). To meet the pharmacopeial quality requirement of being the impurity below 0.1% in the PPIs can only be achieved by several repeated purification, which affects the overall economy of the process/product.

Therefore, there is a need for an alternative solution for controlling the formation of sulphone impurity to an acceptably low level during the process for oxidation of prochiral sulphide compounds, especially the PPIs, owing to the importance of said compounds. Also still exists a need for improved processes for oxidation of prochiral sulphide compounds, especially the PPIs, owing to the importance of said compounds. The present inventors have found alternative processes for the enantioselective oxidation of prochiral sulfide which provides still better optical yields and purity, which process also reduces the sulfone impurity to a minimal level. This becomes the subject of the present invention.

### SUMMARY OF THE INVENTION

Accordingly, the present invention provides new processes for preparation of optically active sulfoxide compounds of Formula I,

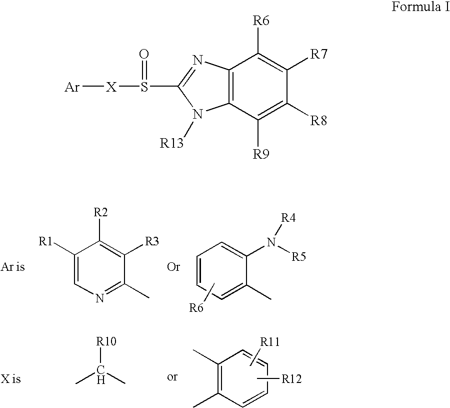

wherein R1, R2 and R3 are the same or different and selected from hydrogen, halogen, nitro, alkyl, alkylthio, alkoxy optionally substituted by fluorine, alkoxyalkoxy, dialkylamino, piperidino, morpholino, halogen, phenylalkyl and phenylalkoxy; R4 and R5 are the same or different and selected from hydrogen, alkyl and aralkyl; R6′ is hydrogen, halogen, trifluoromethyl, alkyl or alkoxy; R6-R9 are the same or different and selected from hydrogen, alkyl, alkoxy, halogen, halo-alkoxy, alkylcarbonyl, alkoxycarbonyl, oxazolyl, trifluoroalkyl, or adjacent groups R6-R9 form ring structures which may be further substituted; R10 is hydrogen or forms an alkylene chain together with R3 and R11 and R12 are the same or different and selected from hydrogen, halogen and alkyl; and R13 is hydrogen or a protective substituent like benzyl, trityl etc.; in the above definitions alkyl groups, alkoxy residues may be branched or straight C1-C9-chains or comprise cyclic alkyl groups, for example cycloalkylalkyl, comprising asymmetric oxidation of prochiral sulphide compounds of Formula II,

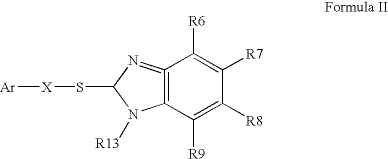

wherein the groups are as defined above, in the presence of a chiral transition metal complex in water and in presence of a base. The characteristic of the invention lies in the enantioselective oxidation that is carried out in the presence of aqueous solvent and in the absence of organic solvent.

In a second aspect of the present invention, a process for preparation of optically active sulphoxides of Formula I is provided which comprises asymmetric oxidation of prochiral sulphides of Formula II in presence of a chiral transition metal complex in water and in presence of a base and catalyst. The catalyst may be selected from sulphoxides or sulphone compounds and or phosphonium compounds. Sulphoxides includes alkyl, aryl or cyclic sulphoxides and especially preferred one is dimethylsulphoxide.

In a third aspect of the present invention, a process for preparation of optically active sulphoxides of Formula I is provided which comprises asymmetric oxidation of prochiral sulphides of Formula II in presence of a chiral transition metal complex in presence of a catalyst. The catalyst may be selected from sulphoxides or sulphone compounds and or phosphonium compounds. Sulfoxides includes alkyl, aryl or cyclic sulphoxides and especially preferred one is dimethylsulphoxide.

In yet another aspect of the present invention, a process for preparation of optically active sulphoxides of Formula I is provided which comprises asymmetric oxidation of prochiral sulphides of Formula II wherein the groups are as defined above, in the presence of a chiral transition metal complex and tritylhydroperoxide. The characteristic of the invention lies in the enantio-selective oxidation that is carried out in the presence of triphenylmethyl hydroperoxide (Also termed as tritylhydroperoxide), which reduces the formation sulphone impurity of Formula III to an acceptably low level, while giving chiral selectivity. One or more of the phenyl rings may be appropriately substituted with an inert group, which may further increase the bulkiness of the oxidizing agent to reduce the sulfone impurity.

Optically active Sulfoxide compounds of Formula I, wherein R2 is a leaving group such as halo, nitro results in chiral sulphoxide compounds which are useful as penultimate intermediates for PPIs. On substitution with appropriate alkoxide according to any known procedure the above proton pump inhibitors of pharmaceutical interest can be obtained.

The transition metal may be selected from the group comprising titanium, zirconium, hafnium and vanadium. The most preferred transition metal is titanium. The transition metal complex may be prepared from a transition metal derivative and a chiral ligand.

Thus, the chiral transition metal complex is prepared by the reaction of transition metal derivative and the complexing chiral ligand, either separately or in the presence of the prochiral sulphide substrate of Formula II at suitable conditions. A suitable reagents and solvent, if required may be used to achieve the complexation of transition metal with the ligand.

The details of one or more embodiments of the inventions are set forth in the description below. Other features, objects and advantages of the inventions will be apparent from the appended examples and claims.

### DETAILED DESCRIPTION OF THE INVENTION

Unless specified otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described. To describe the invention, certain terms are defined herein specifically as follows.

Unless stated to the contrary, any of the words ‘including’, ‘includes’, ‘comprising’ and ‘comprises’ mean ‘including without limitation’ and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

The expressions “pro-chiral sulphide(s)” are used for the sulphides of the corresponding sulphoxides suitable for being prepared by the novel process according to the present invention. If the corresponding sulphide already contains a stereogenic centre in the molecule, such a sulphide is not a pro-chiral compound, but a chiral compound. Since the sulphur atom of such sulphides does not have asymmetry such a compound is referred to as a pro-chiral sulphide in the present specification and appending claims.

The term “omeprazole”, as used herein unless specified otherwise, refers to a racemic mixture of 5-methoxy-2-[(4-methoxy-3,5-dimethyl-2-pyridyl)methylsulfinyl]-1H-benzimidazole and 6-methoxy-2-[(4-methoxy-3,5-dimethyl-2-pyridyl)methylsulfinyl]-1H-benzimidazole in the solid state.

As used herein, “omeprazole” is also represented as 5(6)-methoxy-2-[(4-methoxy-3,5-dimethyl-2-pyridyl)methylsulfinyl]-1H-benzimidazole.

The term “S-omeprazole” or “esomeprazole”, as used herein unless specified otherwise, refers to the S stereoisomer of omeprazole and including its known salts.

The term “R-omeprazole”, as used herein unless specified otherwise, refers to the R stereoisomer of omeprazole.

The terms “S” and “R”, as used herein unless specified otherwise, refer to stereoisomers resulting from the spatial arrangement of groups at a chiral centre, and in the present context, the person of ordinary skill will appreciate that the groups attached with the sulfoxide represents the plane for purposes of determining the configuration.

The term “alkyl” refers to a straight or branched alkyl group having from 1 to 6 carbon atoms. Exemplary alkyl groups include but are not limited to methyl, ethyl, n-propyl, iso-propyl, n-butyl, and iso-butyl.

The term “aryl” refers to an aromatic, optionally fused, carbocycles having from 6 to 20 carbon atoms. Examples of C6-12-aryl include but are not limited to phenyl and napthyl.

The term “alkylaryl” or ‘aralkyl’ refers to an alkyl substituted with one or more aromatic residues, optionally with substituents. Examples of alkylaryl include but are not limited to biphenylmethyl or triphenyl methyl.

The term ‘Enantioselective’ as used herein, refers to the preferential formation of one of the enantiomer to obtain an optically pure compound or optically enriched enantiomeric mixtures in which the ratio of the enantiomers differs.

The term ‘enantiomeric excess’ as used herein, refers generally to the concentration of one stereoisomer that exceeds the concentration of another stereoisomer. Typically, the term is used to characterize the optical purity of an optically active compound that exists in the bulk as two or more stereoisomers. In the present context, the term also refers to the excess of either S- or R-omeprazole over the other that are present in a given enantiomeric enriched mixture of the present invention.

This invention is directed to processes for preparation of sulphoxide compounds that, by virtue of the processes of this invention, are substantially optically pure or optically enriched mixtures of enantiomers. Accordingly, the present invention provides processes for the preparation of optically active substituted pyridinylmethyl sulfinyl-benzimidazoles of the compound of Formula I,

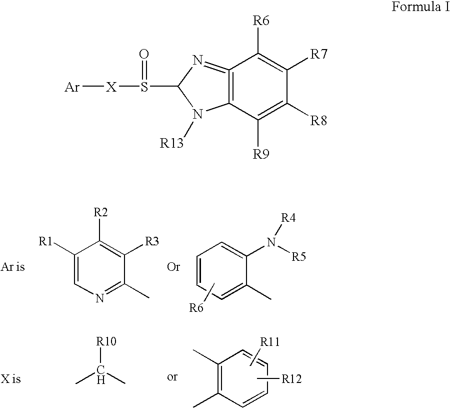

wherein the groups R1 to R13 are as defined previously.

The process comprises enantioselective oxidation of a substituted pyridinylmethyl prochiral sulfide derivative of compound of Formula II,

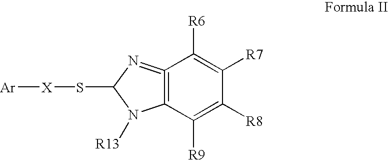

wherein the groups R1 to R13 are as defined above, in the presence of a base in aqueous solvent. The prochiral sulphides may be employed as its stable alkali metal salts during the oxidation. It should be understood that compounds wherein R2 is a leaving group such as halo, nitro are useful as penultimate intermediates for preparing the pharmaceutically valuable PPIs listed above. On substitution with appropriate alkoxide according to any known procedure, the above proton pump inhibitors of pharmaceutical interest can be obtained. The products obtained may thereafter be converted to pharmaceutically acceptable salts thereof by conventional processes either for facilitating purification/isolation or pharmaceutical application.

Substituted optically active sulphoxides prepared by the enantioselective catalytic oxidation process of the present invention may be obtained either in optically active enantiomer or enantiomerically enriched forms, preferably as an optically enriched substituted pyridinylmethyl-sulfinyl-benzimidazole according to the formula I.

Preferably, the process of the present invention provides an enantioselective process for the preparation of optically active or enantiomerically enriched sulfoxides of, especially omeprazole, pantoprazole, rabeprazole and lansoprazole, in free forms or their alkali and/or alkaline earth metal salts, which are proton pump inhibitors useful in the treatment of ulcers.

Thus the present invention provides processes for the preparation of alkali and/or alkaline earth metal salts of an optically active enantiomer or an enantiomerically enriched form of substituted pyridinylmethyl-sulfinyl-benzimidazole, which are of pharmaceutical interest or useful as intermediates for formation/purification of said optically active compounds of Formula I.

The process of the invention is characterized by the enantioselective oxidation of the corresponding prochiral sulphide of Formula H being carried out in the presence of aqueous solvent and a base using the chiral transition metal complex. The oxidation is carried out advantageously in the absence of any organic solvent, but in the presence of water.

In a second aspect of the present invention, a process for preparation of optically active sulphoxides of Formula I is provided which comprises asymmetric oxidation of prochiral sulphides of Formula II in presence of a chiral transition metal complex in water and in presence of a base and catalyst. The catalyst may be selected from sulphoxides or sulphone compounds and or phosphonium compounds. Sulfoxides includes alkyl, aryl or cyclic sulphoxides and especially preferred one is dimethylsulphoxide (DMSO).

In a third aspect of the present invention, a process for preparation of optically active sulphoxides of Formula I is provided which comprises asymmetric oxidation of prochiral sulphides of Formula II in presence of a chiral transition metal complex in presence of a catalyst. The catalyst may be selected from sulphoxides or sulphone compounds and or phosphonium compounds. Sulfoxides includes alkyl, aryl or cyclic sulphoxides. The catalyst, according to the invention, may be selected from sulphoxides compounds. Especially preferred sulfoxide is dimethylsulphoxide. The quantity of the catalyst is not critical for success of oxidation, rather its presence, and it can be in catalytic amounts to molar amounts. The oxidation process, according to this aspect is advantageously carried out in an organic solvent, such as those customarily used, for example, chlorinated hydrocarbons, ethyl acetate, toluene, diethylether, tetrahydrofuran, dioxane, or methyl isobutyl ketone etc. The reaction may be done in presence of water.

In yet another aspect of the present invention, a process for preparation of optically active sulphoxides of Formula I is provided which comprises asymmetric oxidation of prochiral sulphides of Formula II wherein the groups are as defined above, in the presence of a chiral transition metal complex and tritylhydroperoxide. The characteristic of the invention lies in the enantio-selective oxidation that is carried out in the presence of triphenylmethyl hydroperoxide (Also termed as tritylhydroperoxide), which reduces the formation sulphone impurity of Formula III to an acceptably low level, while giving chiral selectivity. One or more of the phenyl rings may be appropriately substituted with an inert group, which may further increase the bulkiness of the oxidizing agent to reduce the sulfone impurity.

In all the above embodiments of the invention, the transition metal may be selected from the group comprising titanium, zirconium, hafnium and vanadium. The most preferred transition metal is titanium. The transition metal complex may be prepared from a transition metal derivative and a chiral ligand.

Suitable transition metal derivative are transition metal (IV) halides or transition metal (IV) alkoxides, or transition metal (IV) acetylacetonates. Examples of halide is chloride, alkoxides are butoxide, tert-butoxide, ethoxide and, in particular, n-propoxide or isopropoxide. The most preferred transition metal derivative is titanium tetrachloride or titanium isopropoxide.

The chiral ligand may be a monodentate, bidentate or polydentate ligand, but preferably a chiral branched or unbranched alkyl diol or an aromatic diol or an aminoalcohol. The preferred chiral diol may be a chiral ester or amide of tartaric acid. Suitable optically pure tartaric acid derivatives are, for example, (+)-L-tartaric acid amides, such as (+)-L-tartaric acid bis-(N,N-diallylamide), (+)-L-tartaric acid bis-(N,N-dibenzylamide), (+)-L-tartaric acid bis-(N,N-diisopropylamide), (+)-L-tartaric acid bis-(N,N-dimethylamide), (+)-L-tartaric acid bis-(N-pyrrolidinamide, (+)-L-tartaric acid bis-(N-piperidinamide), (+)-L-tartaric acid bis-(N-morpholinamide), (+)-L-tartaric acid bis-(N-cycloheptylamide) or (+)-L-tartaric acid bis-(N-4-methyl-N-piperazinamide), or dialkyl (+)-L-tartrate esters such as dibutyl (+)-L-tartrate, di-tert-butyl (+)-L-tartrate, diisopropyl (+)-L-tartrate, dimethyl (+)-L-tartrate and diethyl (+)-L-tartrate, or (−)-D-tartaric acid amides, such as (−)-D-tartaric acid bis-(N,N-diallylamide), (−)-D-tartaric acid bis-(N,N-dibenzylamide), (−)-D-tartaric acid bis-(N,N-diisopropylamide), (−)-D-tartaric acid bis-(N,N-dimethylamide), (−)-D-tartaric acid bis-(N-pyrrolidinamide), (−)-D-tartaric acid bis-(N-piperidinamide), (−)-D-tartaric acid bis-(N-morpholinamide), (−)-D-tartaric acid bis-(N-cycloheptylamide) or (−)-D-tartaric acid bis-(N-4-methyl-N-piperazinamide), or dialkyl (−)-D-tartrate esters such as dibutyl (−)-D-tartrate, di-tert-butyl (−)-D-tartrate, diisopropyl (−)-D-tartrate, dimethyl (−)-D-tartrate and diethyl (−)-D-tartrate or the like.

The chiral transition metal complex can be prepared by the reaction of transition metal derivative and the complexing chiral ligand, either separately or in the presence of the prochiral sulphide substrate of Formula II. A suitable reagents and solvent, if required may be used to achieve the complexation of transition metal with the ligand. For example the titanium isoperoxide is reacted with diethyltartarate directly before addition of the substrate or may be prepared in the presence of the prochiral sulphide of formula II.

The especially preferred titanium complex used advantageously in the present invention is prepared from a chiral diethyltartarate and a titanium(IV) compound, preferably titanium(IV) alkoxide in the presence or absence of water. An especially preferred titanium(IV) alkoxide is titanium(IV) isopropoxide or n-propoxide. When the titanium complex is prepared by reacting titanium tetra chloride with a chiral ligand, a base is advantageously used in the process.

The base used in the enantioselective oxidation may be an inorganic or an organic base; examples of organic base include trimethylamine, triethylamine, tributylamine, tri isopropylamine, diisopropylethylamine, pyridine, morpholine, DBU (1,8-diazabicyclo-[5.4.0]-undec-7-ene), DBN (1,5-diazabicyclo-[4.3.0]-non-5-ene), 4-dimethylamino pyridine and mixtures thereof. Examples of inorganic bases include alkali metal carbonate, bicarbonate, hydroxide and mixtures thereof. Examples of alkali metal carbonates include lithium carbonate, sodium carbonate and potassium carbonate. Examples of alkali metal bicarbonates include sodium bicarbonate and potassium bicarbonate. Examples of alkali metal hydroxides include sodium hydroxide and potassium hydroxide. Organic bases are preferred for this application and especially suitable bases are amines, preferably triethylamine or N,N-diisopropylethylamine. The amount of base added to the reaction mixture is not very critical but should be adjusted with respect to the respective substrates or can be established by trial.

The metal complex may be added to the reaction mixture containing prochiral sulfide. Alternately, the reaction mixture containing prochiral sulfide may be added to the metal complex. The amount of the chiral titanium complex is not critical to the success. Even in catalytic quantities of chiral titanium complexes are sufficient to give excellent stereoselective oxidation of the sulphide and an optimum amount may be worked out by trial in a particular substrate.

Suitable oxidizing agents are any oxidizing agents customarily used for the synthesis of substituted sulphenyl compounds of Formula I, where particular mention may be made of hydroperoxides, such as, for example, alkyl hydro peroxide, arylhydroperoxides and aryl alkyl hydro peroxide. The aryl alkyl hydro peroxide may be cumene hydro peroxide or trityl hydroperoxide. Especially preferred alkyl hydroperoxide is ter-butyl hydroperoxide. The most preferred hydroperoxide is a trityl hydroperoxide because it significantly reduces the amount of sulphone impurity during the oxidation reaction compared with other hydroperoxides. In general, 0.50 to molar excess oxidation equivalents, preferably 0.99-1.3 equivalents, of the oxidizing agent are used.

The oxidation is carried out at a temperature, for example between 20-70 degrees, preferably carried at room temperature or just above room temperature. Lower temperature results in long reaction times and a suitable temperature range is chosen depending on the stability/decomposition of the compounds.

In the process of the present invention, the preparation of the chiral titanium complex is performed at a temperature between 20-70 degrees and optionally in presence of the prochiral sulfide substrate. The transition metal complex preparation time is approximately from 0-1.5 hours. Then the oxidizing agent is introduced in the reaction. The enantioselective oxidation time varies depending on the reaction temperature and type of the pro-chiral sulphide, and usually completes within 10 minutes to 2.5 hours. In some cases prolonged reaction is not advisable, as the product/starting sulfide degrades during reaction.

If the process is carried out in a suitable manner, the optically pure sulphinyl compound of formula I is obtained in an optical purity of >70%, preferably greater than 80%, and more preferably greater than 95%. By further steps, such as, for example, pH-controlled reprecipitation and/or recrystallization in a suitable solvent, it is possible to further increase the optical purity to even greater than 99.5%. Reprecipitation is carried out via intermediate preparation of suitable salts, such as, for example, potassium, sodium, calcium or barium salt.

The obtained crude product may be extracted in an organic solvent. It may also be crystallized in an organic or aqueous solvent resulting in an optically pure product. A suitable metal salt of the compound of Formula I may be obtained by treating the crude product with alkali or alkaline earth metal source. followed by crystallisation of the formed salt in a solvent which may result in a product with an improved optical purity.

Thus process of the present invention is applicable for the preparation of an optically active alkali and/or alkaline earth metal salt of substituted sulphinylbenzimidazole by treating the optically active substituted sulphinylbenzimidazole compound of Formula I, obtained by enantioselective catalytic oxidation by treating with an alkali and/or alkaline earth metal source. The alkali or alkaline earth metal source may be selected from Na⁺, Li⁺, Mg⁺², Ca⁺² and Ba⁺² salts such as bicarbonates, carbonates, hydrides, hydroxides, halides, sulphates, alkoxides and oxides. In particular, sodium hydroxide, sodium methoxide, sodium ethoxide, potassium hydroxide, potassium tertiarybutoxide, barium hydroxide, lithium hydroxide, magnesium hydroxide, magnesium chloride, calcium chloride may be used.

The process of the present invention further includes the optional steps of isolating the alkali or alkaline earth metal salts of the optically active substituted pyridinylmethyl-sulfinyl-benzimidazole compound of Formula 1 by solvent evaporation with or without vacuum, followed by addition of the organic solvent and/or an antisolvent and filtering the product, and drying, as required. It may be again purified by similar or any known procedures to either increase the optical purity or to reduce the sulphone content, for examples, esomeprazole potassium is purified from alcohol such as methanol to reduce the sulphone impurity.

The products described above can be further processed if desired. Alkali or alkaline earth metal salts of benzimidazole compounds may be exchanged with another alkali or alkaline earth metal salts to prepare a desired PPI for pharmaceutical application. For example, esomeprazole sodium or potassium can be converted into esomeprazole magnesium.

The PPIs obtained by the process of the present invention, for example, esomeprazole magnesium, may be formulated into a dosage form, e.g., tablet, capsule, etc., by combining with one or more pharmaceutically acceptable excipients using known techniques. The resulting dosage form may include a suitable amount of the active ingredient. For example, the resulting dosage form may contain between 5 and 50 mg of esomeprazole magnesium. Further, the dosage form may be immediate release or extended release. The dosage forms may be administered to a mammal in need, as proton pump inhibitors useful for treating ulcers.

The following examples are presented to further explain the invention with experimental conditions, which are purely illustrative and are not intended to limit the scope of the claimed invention.

### EXAMPLES

### Example 1

### Oxidation Process in Presence of Water Solvent

In a 100 ml flask, 7.5 gm diethyl (−)-D-tartrate, 1.17 gm diisopropyl ethyl amine, 5.16 gm titanium(IV) isopropoxide and 0.13 ml water were taken at room temperature, and the mixture was heated to 65-70 degrees for 1 hour. After cooling to room temperature, 10 gm 5-methoxy[(2-(4-methoxy)-3,5-dimethyl-2-pyridinyl]methylsulfenyl]-1H-benzimidazole (also termed as pyrmetazole or PMT) was added and heated until a clear solution was obtained. The mixture then stirred for 0.5 hours at heating and cooled to room temperature. 6.9 gm Cumene hydroperoxide was added slowly. After addition, the reaction was monitored by HPLC for the following with the results listed below:

| | Sulfide | | | enantiomeric |
|---|---|---|---|---|
| Analysis | substrate | sulphoxide | sulphone | S/R |
| Results (%) | 9.69 | 81.87 | 7.67 | 83.3/16.64 |

### Isolation:

To the reaction mixture 30 ml methanol, 0.1 gm KI, and 2.74 gm potassium methoxide was added and stirred. To this 30 ml toluene was added and filtered to obtain a crude esomeprazole potassium salt. Yield 65-70%. The analysis of crude product is as follows:

| | Sulfide | | | enantiomeric |
|---|---|---|---|---|
| Analysis | substrate | sulphoxide | sulphone | S/R |
| Results (%) | 0.20 | 97.18 | 2.70 | 99.7/0.30 |

### Purification:

In a 1 Litre flask, 100 gm crude esomeprazole potassium in 500 ml methanol were taken and heated to reflux. After 1 hour reflux, the mass was cooled to 0-5 degree, filtered, washed with chilled methanol. The wet product again treated with methanol as per above procedure and received 83 gm (dried) esomeprazole potassium salt. HPLC analysis shows: Esomeprazole e.e 99.5% and sulfone 0.05%.

### Preparation of Esomeprazole Magnesium.

In a 250 ml flask, 20 gm esomeprazole potassium (pure) and 120 ml water were taken and stirred until a clear solution was formed. To this 20 ml Toluene was added and separated the organic layer. To the aqueous layer containing esomeprazole potassium salt, 40 ml methanol and 7.7 gm Magnesium sulphate in 20 ml water was added. The suspension is stirred for 1 hour and filtered, washed with 100 ml waster, and dried to obtain 14.5 gm of esomeprazole magnesium. HPLC analysis shows: Esomeprazole e.e. 99.93% and sulfone<0.05%.

### Example 2

### Oxidation Process in Presence of Base, Catalyst & Water Solvent

In a 100 ml flask, 7.5 gm diethyl (−)-D-tartrate, 1.17 gm diisopropyl ethyl amine, 5.16 gm titanium(IV) isopropoxide and 0.13 ml water were taken at room temperature, and the mixture was heated to 65-70 degrees for 1 hour. After cooling to room temperature, 10 gm 5-methoxy[(2-(4-methoxy)-3,5-dimethyl-2-pyridinyl]methylsulfenyl]-1H-benzimidazole (also termed as pyrmetazole or PMT) was added and heated until a clear solution was obtained. The mixture then stirred for 0.5 hours at heating and cooled to room temperature. 0.71 gm DMSO and 6.9 gm Cumene hydroperoxide was added slowly. After addition, the reaction was monitored by HPLC for the following with the results listed below:

| | Sulfide | | | enantiomeric |
|---|---|---|---|---|
| Analysis | substrate | sulphoxide | sulphone | S/R |
| Results (%) | 16.33 | 75 | 6.83 | 81/19 |

### Isolation:

To the reaction mixture 30 ml methanol, 0.1 gm KI, and 2.74 gm potassium methoxide was added and stirred. To this 30 ml toluene was added and filtered to obtain a crude product. The analysis of crude product is as follows:

| | Sulfide | | | enantiomeric |
|---|---|---|---|---|
| Analysis | substrate | sulphoxide | sulphone | S/R |
| Results (%) | — | 97.12 | 2.5 | 99.76/0.24 |

### Example 3

### Reaction in Presence of Water Solvent and Absence of Organic Solvent

In a 100 ml flask, 7.5 gm diethyl (−)-D-tartrate and 5.16 gm titanium(IV) isopropoxide and 0.13 ml water were taken at room temperature, and the mixture was heated to 65-70 degrees for 1 hour. After cooling to room temperature, 10 gm 5-methoxy[(2-(4-methoxy)-3,5-dimethyl-2-pyridinyl]methylsulfenyl]-1H-benzimidazole was added and heated until a clear solution was obtained. The mixture then stirred for 0.5 hours at heating and cooled to room temperature. 6.9 gm Cumene hydroperoxide was added slowly. After addition, the reaction was monitored by HPLC for the following with the results listed below:

| | Sulfide | | | enantiomeric |
|---|---|---|---|---|
| Analysis | substrate | sulphoxide | sulphone | excess S/R |
| Results (%) | 22.98% | 61.05% | 5.53% | 78.86/21.44 |

### Comparative Example 1

### With Water & Organic Solvent Alone

In a 100 ml flask, 7.5 gm diethyl (−)-D-tartrate, 20 ml methylene chloride, 5.16 gm titanium(IV) isopropoxide and 0.13 gm water were taken at room temperature, and the mixture was heated to 65-70 degrees for 1, hour. After cooling to room temperature, 10 gm PMT was added and heated until a clear solution was obtained. The mixture then stirred for 0.5 hours at heating and cooled to room temperature. 6.9 gm Cumene hydroperoxide was added slowly. After addition, the reaction was monitored by HPLC for the following with the results listed below:

| | Sulfide | | | enantiomeric |
|---|---|---|---|---|
| Analysis | substrate | sulphoxide | sulphone | S/R |
| Results (%) | 38.2% | 52.20% | 1.57% | 63/37 |

### Example 4

### Preparation of Tritylhydroperoxide

In a 1 L flask, 50 gm trityl chloride and toluene (250 ml) were taken and stirred for 30 minutes at room temperature. After cooling, 17.92 gm sodium bicarbonate and 81.4 gm H₂O₂ (45%) was added and the pH of the solution adjusted to 4-5. After stirring for 30 minutes, 50 ml water was added, layer separated and aqueous layer extracted with 100 ml toluene. The organic layer was dried using anhydrous sodium sulphate, filtered to get 290 ml of titylhydroperoxide in toluene solution. HPLC purity 85-90%, and yield=98%.

### Example 5

a) Oxidation: In a 1 L flask under nitrogen atmosphere, 50 gm 5-methoxy[(2-(4-methoxy)-3,5-dimethyl-2-pyridinyl]methylsulfenyl-1H-benzimidazole (also termed as pyremetazole or PMT) was mixed with 160 ml toluene and heated to 65-70 degrees. To this mixture 18.8 gm diethyl (−)-D-tartrate and 45 ml toluene, 5.9 gm diisopropyl ethyl amine, 12.9 gm titanium(IV) isopropoxide were added and continue to stir at 60-65 degree for 1 hour. To this mixture after cooling, trityl hydroperoxide solution in toluene (279 ml having 47 gm tritylhydroperoxide) was added room temperature. The mixture was maintained under stirring for 3 hours at room temperature. The reaction mixture was analyzed for sulphoxide and sulfone. The sulfone content was less than 1%.

To the reaction mixture 30 ml methanol, 0.1 gm KI, and 2.74 gm potassium methoxide was added and stirred. The mixture cooled to 20 degree and filtered, washed with 150 ml Toluene-methanol mixture, followed by 50 ml Methanol. Yield=83%. The HPLC analysis of product shows:

Esomeprazole (as potassium salt) 99.21% (99% ee) and sulfone content 0.59%.

b) Purification of esomeprazole potassium: In a 1 Litre flask, 100 gm crude esomeprazole potassium in 500 ml methanol were taken and heated to reflux. After 1 hour reflux, the mass was cooled to 0-5 degree, filtered, washed with chilled methanol, and dried to obtain 90 gm of esomeprazole potassium. HPLC analysis shows: Esomeprazole ee 99.5% and sulfone 0.05%.

### Example 6

The example 2 was repeated by varying the molar amounts of trityl hydroperoxide and the results are summarized below:

### Reaction Mass:

| | | Molar | | | |
|---|---|---|---|---|---|
| Serial | Stage of HPLC | amount of | Reaction | Esomeprazole | Sulfone |
| No. | analysis | THP | time | content | content |
| A | Reaction mass | | | | |
| 1 | | 0.85 | 6 hours | 79.5 | 0.55 |
| 2 | | 0.95 | 3 hours | 88.8 | 1.06 |
| 3 | | 1.25 | 3 hours | 92.07 | 2.36 |
| B | Crude product | | | | |
| 1 | | 0.85 | | 97.78 | 0.32 |
| 2 | | 0.95 | | 99.21 | 0.59 |
| 3 | | 1.25 | | 98.50 | 1.23 |
| C | Purification | | | | |
| 1 | | 0.85 | | 99.70 | 0.03 |
| 2 | | 0.95 | | 99.60 | 0.05 |
| 3 | | 1.25 | | 99.60 | 0.30 |

Purification of the above obtained product was carried out in THF in place of methanol and the results are as follows:

| Serial | Stage of HPLC | Esomeprazole | Sulfone |
|---|---|---|---|
| No. | analysis | content | content |
| A | Crude | 99.10 | 0.67 |
| | Pure | 99.70 | 0.08 |
| B | Crude | 99.23 | 0.25 |
| | Pure | 92.80 | 0.015 |

### Example 7

### Preparation of Esomeprazole Magnesium

In a 250 ml flask, 20 gm esomeprazole potassium (pure) and 120 ml water were taken and stirred until a clear solution was formed. To this 20 ml Toluene was added and separated the organic layer. To the aqueous layer containing esomeprazole potassium salt, 40 ml methanol and 7.7 gm Magnesium sulphate in 20 ml water was added. The suspension is stirred for 1 hour and filtered, washed with 100 ml waster, and dried to obtain 14.5 gm of esomeprazole magnesium. HPLC analysis shows: Esomeprazole e.e. 99.93% and sulfone<0.05%.

### Example 8

In a 1 L flask under nitrogen atmosphere, 50 gm pyremetazole was mixed with 160 ml toluene and heated to 65-70 degrees. To this mixture 18.8 gm diethyl (+)-L-tartrate and 45 ml toluene, 12.9 gm titanium(IV) isopropoxide were added and continue to stir at 60-65 degree for 1 hour. To this mixture after cooling, 1.5 gm DMSO and trityl hydroperoxide solution in toluene (279 ml having 27% tritylhydroperoxide content) was added room temperature. The mixture was maintained under stirring for 3 hours at room temperature. The reaction mixture was analyzed for sulphoxide and sulfone. The sulfone content was less than 1.0%.

To the reaction mixture 30 ml methanol, 0.1 gm KI, and 2.74 gm potassium methoxide was added and stirred. The mixture cooled to 20 degree and filtered, washed with 150 ml Toluene-methanol mixture, followed by 50 ml Methanol. Yield 80%. The HPLC analysis of product shows:

Esomeprazole (as potassium salt) 99.3%, e.e. 99% and sulfone content 0.90%.

### Example 9

### Preparation of Esomeprazole Free Base

In a 500 ml flask, 18.8 gm diethyl (−)-D-tartrate, 12.9 gm titanium(IV) isopropoxide and 0.33 ml water were taken at room temperature, and 50 gm 5-methoxy[(2-(4-methoxy)-3,5-dimethyl-2-pyridinyl]methylsulfenyl]-1H-benzimidazole (also termed as pyrmetazole or PMT) was added and the mixture was heated to 70-75 degrees for 1 hour. After cooling to 40 degrees, 3 gm diisopropyl ethyl amine and 34.6 gm (80%) cumene hydroxide were added and maintained mixture under stirring. The reaction was monitored, after completion of reaction 100 ml toluene and 31.6 gm NaOH solution were added to the mixture. The mixture then stirred for 0.5 hours at heating and then cooled to room temperature and further to 0-5 degree Celsius. Layers were separated and toluene layer washed with water. The aqueous layer neutralized with conc. Hydrochloric acid and esomeprazole free base was extracted using dichloromethane. The dichloromethane layers, dried, and evaporated to obtain esomeprazole free base.

### Example 10

### Conversion of Esomeprazole Free Base to Barium Salt

Esomeprazole free base 52.1 gm was suspended in 5 volume methanol. A solution of barium hydroxide (prepared by dissolving 49 gm barium hydroxide in 8 volume methanol) was added to the esomeprazole solution in methanol. The mixture was stirred overnight and then filtered. The precipitate was dried to get 50 gm esomeprazole barium.

### Example 11

### Preparation of Esomeprazole Magnesium

Esomeprazole barium 10 gm was mixed with 20 volume of water, and heated to 70 degrees Celsius for 1 hour. The mixture was filtered and 3.7 gm magnesiumchloride in 1 volume water was added. Then the mixture was cooled to 30 degrees and maintained for 24 hours. The precipitate obtained was filtered, washed with water and dried to obtain 7.4 gm esomeprazole magnesium dihydrate. Esomeprazole e.e. 99.92%, sulfone<0.05% by HPLC.

### Comparative Example 2

With the application of Cumene hydroperoxide & tertiary butyl hydroperoxide in the place of tritylhydroperoxide as the oxidizing agent in example 5.

The reaction mass analysis results are as follows:

1. Results of Cumene Hydroperoxide as Oxidizing Agent.

| | No. of moles | | | | |
|---|---|---|---|---|---|
| | of oxidizing | Reaction mass | | Crude isolated | |
| Serial | agent | Esomeprazole | Sulfone | Esomeprazole | Sulfone |
| No. | w.r.t. PMT | content | content | content | content |
| A | | | | | |
| 1 | 0.97 | 82.0% | 2.05 | 97.80 | 1.48 |
| 2 | 1.15 | 93.0 | 3.07 | 97.70 | 2.60 |
| 3 | 1.25 | 95.05 | 3.90 | 97.90 | 2.96 |

2. Results of Tritylhydroperoxide as Oxidizing Agent

| | No. of moles | | | | |
|---|---|---|---|---|---|
| | of oxidizing | Reaction mass | | Crude isolated | |
| Serial | agent | Esomeprazole | Sulfone | Esomeprazole | Sulfone |
| No. | w.r.t. PMT | content | content | content | content |
| A | | | | | |
| 1 | 0.85 | 79.5 | 0.55 | 97.78 | 0.32 |
| 2 | 0.95 | 88.8 | 1.06 | 99.21 | 0.59 |
| 3 | 1.25 | 92.07 | 2.36 | 98.50 | 1.23 |

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1. A process for preparation of optically active sulfoxide compounds of Formula I, 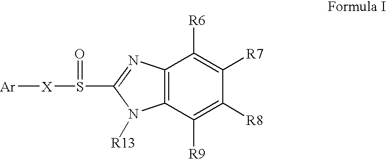
Ar is 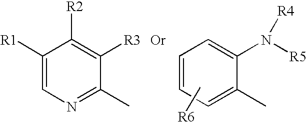
X is 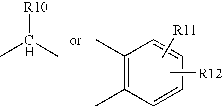
wherein R1, R2 and R3 are the same or different and selected from hydrogen, halogen, nitro, alkyl, alkylthio, alkoxy optionally substituted by fluorine, alkoxyalkoxy, dialkylamino, piperidino, morpholino, halogen, phenylalkyl and phenylalkoxy; R4 and R5 are the same or different and selected from hydrogen, alkyl and aralkyl; R6′ is hydrogen, halogen, trifluoromethyl, alkyl or alkoxy; R6-R9 are the same or different and selected from hydrogen, alkyl, alkoxy, halogen, halo-alkoxy, alkylcarbonyl, alkoxycarbonyl, oxazolyl, trifluoroalkyl, or adjacent groups R6-R9 form ring structures which may be further substituted; R10 is hydrogen or forms an alkylene chain together with R3 and R11 and R12 are the same or different and selected from hydrogen, halogen and alkyl; and R13 is hydrogen or a protective substituent; the method comprising the step of asymmetrically oxidizing prochiral sulphide compounds of Formula II, 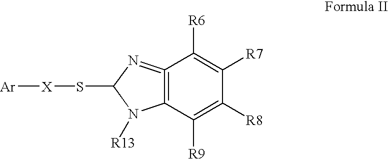
wherein the groups are as defined above, in the presence of a chiral transition metal complex in water and in presence of a base.

2. A process as claimed in claim 1, wherein the oxidizing step is further in presence of a catalyst.

3. A process as claimed in claim 1, wherein the base is selected from organic base or an inorganic base.

4. A process as claimed in claim 3, wherein the base is diisopropylethylamine.

5. A process as claimed in claim 2, wherein the catalyst is a compound selected from the group consisting of sulphoxides, sulphone compounds, and phosphonium compounds.

6. A process as claimed in claim 5, wherein the sulfoxide is dimethylsulphoxide.

7. A process as claimed in claim 1, wherein the oxidizing step is carried out in presence of a hydroperoxide.

8. A process as claimed in claim 7, wherein the hydroperoxide is tertiary butyl hydroperoxide, cumenehydroperoxide or tritylhydroperoxide.

9. A process as claimed in claim 8, wherein the oxidizing step is carried out in presence of tritylhydroperoxide.

10. A process as claimed in claim 1, wherein R2 represents a leaving group, the process further comprising substitution with appropriate alkoxide.

11. A process as claimed in claim 1, wherein the transition metal is titanium, zirconium, hafnium and vanadium.

12. A process as claimed in claim 11, wherein the transition metal is titanium.

13. A process as claimed in claim 1, wherein the transition metal complex is obtained from a transition metal derivative and a chiral ligand.

14. A process as claimed in claim 13, wherein the chiral ligand is a monodentate, a bidentate or a polydentate ligand, each of which is selected from the group consisting of a chiral branched or unbranched alkyl diol, an aromatic diol and an aminoalcohol.

15. A process as claimed in claim 13, wherein the chiral ligand is a chiral ester or amide of tartaric acid selected from the group consisting of (+)-L-tartaric acid amides, dialkyl (+)-L-tartrate esters (−)-D-tartaric acid amides, and dialkyl (−)-D-tartrate esters.

16. A process as claimed in claim 1, wherein the optically active sulphoxide compound of Formula I is 5-methoxy-2-[(4-methoxy-3,5-dimethyl-2-pyridinyl)methylsulphinyl]-1H-benzimidazole (omeprazole), (S)-5-methoxy-2-[(4-methoxy-3,5-dimethyl-2-pyridinyl)methylsulphinyl]-1H-benzimidazole (esomeprazole), 5-difluoromethoxy-2-[(3,4-dimethoxy-2-pyridinyl)methylsulphinyl]-1H-benzimidazole (pantoprazole), 2-[3-methyl-4-(2,2,2-trifluoroethoxy)-2-pyridinyl)methylsulphinyl]-1H-benzimidazole (lansoprazole), 2-([4-(3-methoxypropoxy)-3-methylpyridin-2-yl]methylsulphinyl)-1H-benzimidazole (rabeprazole), or 5-methoxy-2-((4-methoxy-3,5-dimethyl-2-pyridylmethyl) sulphinyl)-1H-imidazo (4,5-b) pyridine (tenatoprazole).

17. A process as claimed in claim 1, further comprising the step of purifying the optically active sulphoxide compound of Formula I by forming salts of calcium, barium, sodium or potassium.

18. A process as claimed in claim 17, wherein the optically active sulphoxide compound is esomeprazole.

19. A process as claimed in claim 18, wherein further comprising the step of converting the optically pure salts of calcium, barium, sodium, and potassium to magnesium salt of esomeprazole.

20. A process as claimed in claim 1, wherein the protective substituent of R13 is benzyl or trityl.

21. A process as claimed in claim 10, wherein the leaving group is halo or nitro.

22. A process as claimed in claim 15, wherein the tartaric acid amides is selected from the group consisting of (+)-L-tartaric acid bis-(N,N-diallylamide), (+)-L-tartaric acid bis-(N,N-dibenzylamide), (+)-L-tartaric acid bis-(N,N-diisopropylamide), (+)-L-tartaric acid bis-(N,N-dimethylamide), (+)-L-tartaric acid bis-(N-pyrrolidinamide, (+)-L-tartaric acid bis-(N-piperidinamide), (+)-L-tartaric acid bis-(N-morpholinamide), (+)-L-tartaric acid bis-(N-cycloheptylamide), and (+)-L-tartaric acid bis-(N-4-methyl-N-piperazinamide).

23. A process as claimed in claim 15, wherein the dialkyl (+)-L-tartrate esters is selected from the group consisting of dibutyl (+)-L-tartrate, di-tert-butyl (+)-L-tartrate, diisopropyl (+)-L-tartrate, dimethyl (+)-L-tartrate, and diethyl (+)-L-tartrate.

24. A process as claimed in claim 15, wherein the (−)-D-tartaric acid amides is selected from the group consisting of (−)-D-tartaric acid bis-(N,N-diallylamide), (−)-D-tartaric acid bis-(N,N-dibenzylamide), (−)-D-tartaric acid bis-(N,N-diisopropylamide), (−)-D-tartaric acid bis-(N,N-dimethylamide), (−)-D-tartaric acid bis-(N-pyrrolidinamide), (−)-D-tartaric acid bis-(N-piperidinamide), (−)-D-tartaric acid bis-(N-morpholinamide), (−)-D-tartaric acid bis-(N-cycloheptylamide), and (−)-D-tartaric acid bis-(N-4-methyl-N-piperazinamide.

25. A process as claimed in claim 15, wherein the dialkyl (−)-D-tartrate esters is selected from the group consisting of dibutyl (−)-D-tartrate, di-tert-butyl (−)-D-tartrate, and diisopropyl (−)-D-tartrate, dimethyl (−)-D-tartrate, and diethyl (−)-D-tartrate.
